(19) United States
(12) Patent Application Publication
(10) Pub. No.: US 2026/0178826 A1
(43) Pub. Date: Jun. 25, 2026

(54) SYSTEM, APPARATUS, AND METHOD FOR GENERATING MEDICAL QUERY RESPONSES

(71) Applicant: Vikas Khurana, South Abington Township, PA (US)

(72) Inventor: Vikas Khurana, South Abington Township, PA (US)

(21) Appl. No.: 19/000,498

(22) Filed: Dec. 23, 2024

(51) Int. Cl. G06F 40/194; G06F 16/334; G06F 21/62; G16H 10/60

(52) U.S. Cl. CPC G06F 40/194; G06F 16/3347; G06F 21/6245; G16H 10/60

(57) ABSTRACT

Disclosed is a method for generating a response to a medical query. The method includes receiving a medical query from a user device, retrieving first data including unstructured patient-based information and second data including structured environment-based information from a database, applying a governance layer to remove public health information, vectorizing the data, generating a response using a large language model (LLM), comparing the response with the vectorized data to determine a plagiarism level, and transmitting the response to the user device when the plagiarism level is within a predetermined range. The method further includes determining relations between the data and query, preparing and organizing data chunks, and modifying the query based on user-defined metadata. An apparatus and system including the user device and apparatus for implementing the method are also disclosed.

### FIELD OF INVENTION

The present disclosure relates to medical information systems, and more particularly to a system, apparatus, and method for generating responses to medical queries using artificial intelligence and natural language processing techniques.

### BACKGROUND

The increasing prevalence of various diseases has heightened the need for early diagnosis, leading to increased testing and generating large amounts of data for patients. This requires management of medical data for a large population. The learnings for the population level data need to be applied to an individual patient. Managing medical records of many individuals on a large scale poses significant challenges. Due to the complexity involved in maintaining these records, accessing specific information can be difficult.

Conventional techniques of data management and prompt provisions are often unreliable due to uncertainties regarding the source or origin of the prompt. Typically, the source of prompt and prompt response is unverified, leading to questions about the accuracy of the information provided by these traditional methods. In the context of managing medical data, this data is frequently displayed or uploaded to websites (web) or servers, compromising the security of the medical data. Furthermore, conventional methods often fail to understand the actual context of the query or concern, resulting in irrelevant prompts being provided in response to specific queries. The system should ensure data privacy and a bias-free environment, with query paths and responses that are explainable and traceable, referencing material for transparency. Given the LLM's human-like response to queries based on provided information, the results may change with selected reference materials. Traceability is essential in distinguishing factual errors from AI hallucination errors in dynamically changing healthcare scenarios.

Therefore, there exists a need for an improved approach that addresses the aforementioned shortcomings of traditional techniques in managing data and providing prompts.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In an aspect of the present disclosure, a method for generating a response to a medical query is disclosed. The method includes receiving a medical query from a user device by processing circuitry. The method further includes retrieving first data and second data from a database by the processing circuitry. The first data comprises unstructured patient-based information and the second data comprises structured environment-based information. The method further includes applying a governance layer to the first data and the second data to remove public health information by the processing circuitry. The method further includes vectorizing the first data and the second data to prepare vectorized data by the processing circuitry. The method further includes generating a response to the medical query based on the vectorized data using a large language model (LLM) by the processing circuitry. The method further includes comparing the generated response with the vectorized data to determine a plagiarism level by the processing circuitry. The method further includes transmitting the generated response by the processing circuitry to the user device when the plagiarism level is within a predetermined range. The method further includes transmitting the generated response with a caution score by the processing circuitry to the user device such that the caution score is generated when the plagiarism level is beyond the predetermined range.

In some aspects of the present disclosure, the method further includes determining one or more relations between the first data, the second data, and the medical query prior to vectorizing the first data and the second data by the processing circuitry.

In some aspects of the present disclosure, for vectorizing the first data and the second data, the method further includes preparing a plurality of chunks from the first data and the second data and organizing the plurality of chunks based on their relation to each other by the processing circuitry.

In some aspects of the present disclosure, the method further includes modifying the medical query based on user-defined metadata received from the user device by the processing circuitry.

In some aspects of the present disclosure, prior to transmitting the generated response with the caution score, the method further includes revising a portion of the generated response that is non-overlapped with the vectorized data when the plagiarism level is beyond the predetermined range by the processing circuitry, and comparing the revised generated response with the vectorized data to determine the plagiarism level by the processing circuitry.

In some aspects of the present disclosure, the method includes analyzing the generated response to identify recommendations for operational procedures or invasive/minimally invasive diagnostic procedures by the processing circuitry. The method further includes generating a link for accessing a consent management platform based on the identified recommendations by the processing circuitry. The method further includes generating a risk tolerance score based on responses provided by the patient over the consent management platform to questions related to risks associated with the identified recommendations by the processing circuitry. The method further includes generating a risk matrix representing probability and severity of risk events associated with the recommended operational procedures or invasive/minimally invasive diagnostic procedures by the processing circuitry. The method further includes stratifying risk to the patient based on the risk tolerance score and the risk matrix by the processing circuitry. The method further includes generating a set of tasks and a questionnaire for the patient based on the stratified risk by the processing circuitry. The method furthermore includes generating a consent form to receive consent of the patient for the operational procedures or the invasive/minimally invasive diagnostic procedure upon completion of the set of tasks and the questionnaire by the processing circuitry.

In an aspect of the present disclosure, an information processing apparatus for generating a response to a medical query is disclosed. The information processing apparatus includes a database configured to store first data comprising unstructured patient-based information and second data comprising structured environment-based information. The information processing apparatus includes processing circuitry configured to receive a medical query from a user device coupled to the information processing apparatus. The processing circuitry retrieves the first data and the second data from the database. The processing circuitry applies a governance layer to the first data and the second data to remove public health information. The processing circuitry vectorizes the first data and the second data to prepare vectorized data. The processing circuitry generates a response to the medical query based on the vectorized data using a large language model (LLM). The processing circuitry compares the generated response with the vectorized data to determine a plagiarism level. The processing circuitry transmits the generated response to the user device when the plagiarism level is within a predetermined range. The processing circuitry transmits the generated response with a caution score to the user device such that the caution score is generated when the plagiarism level is beyond the predetermined range.

In some aspects of the present disclosure, the processing circuitry determines one or more relations between the first data, the second data, and the medical query prior to vectorizing the first data and the second data.

In some aspects of the present disclosure, to vectorize the first data and the second data, the processing circuitry prepares a plurality of chunks from the first data and the second data. The processing circuitry further organizes the plurality of chunks based on their relation to each other.

In some aspects of the present disclosure, to organize the plurality of chunks, the processing circuitry arranges the plurality of chunks into a virtual grid based on their relevancy to each other.

In some aspects of the present disclosure, the processing circuitry modifies the medical query based on user-defined metadata received from the user device.

In some aspects of the present disclosure, prior to transmit the generated response with the caution score, the processing circuitry revises a portion of the generated response that is non-overlapped with the vectorized data when the plagiarism level is beyond the predetermined range. The processing circuitry further compares the revised generated response with the vectorized data to determine the plagiarism level.

In some aspects of the present disclosure, the processing circuitry analyzes the generated response to identify recommendations for operational procedures or invasive/minimally invasive diagnostic procedures. Further, the processing circuitry generates a link for accessing a consent management platform based on the identified recommendations. Further, the processing circuitry generates a risk tolerance score based on responses provided by the patient over the consent management platform to questions related to risks associated with the identified recommendations. Further, the processing circuitry generates a risk matrix that represents probability and severity of risk events associated with the recommended operational procedures or invasive/minimally invasive diagnostic procedures. Further, the processing circuitry stratifies risk to the patient based on the risk tolerance score and the risk matrix. Further, the processing circuitry generates a set of tasks and a questionnaire for the patient based on the stratified risk. Further, the processing circuitry generates a consent form to receive consent of the patient for the operational procedures or the invasive/minimally invasive diagnostic procedure upon completion of the set of tasks and the questionnaire.

In an aspect of the present disclosure, a system for generating a response to a medical query is disclosed. The system includes a user device configured to transmit a medical query and receive a response. The system includes an information processing apparatus comprising a database configured to store first data comprising unstructured patient-based information and second data comprising structured environment-based information. The information processing apparatus includes processing circuitry configured to receive the medical query from the user device. The processing circuitry retrieves the first data and the second data from the database. The processing circuitry applies a governance layer to the first data and the second data to remove public health information. The processing circuitry vectorizes the first data and the second data to prepare vectorized data. The processing circuitry generates a response to the medical query based on the vectorized data using a large language model (LLM). The processing circuitry compares the generated response with the vectorized data to determine a plagiarism level. The processing circuitry transmits the generated response to the user device when the plagiarism level is within a predetermined range. The processing circuitry transmits the generated response with a caution score to the user device such that the caution score is generated when the plagiarism level is beyond the predetermined range.

In some aspects of the present disclosure, the processing circuitry determines one or more relations between the first data, the second data, and the medical query prior to vectorizing the first data and the second data.

In some aspects of the present disclosure, to vectorize the first data and the second data, the processing circuitry prepares a plurality of chunks from the first data and the second data. The processing circuitry further organizes the plurality of chunks based on their relation to each other.

In some aspects of the present disclosure, to organize the plurality of chunks, the processing circuitry arranges the plurality of chunks into a virtual grid based on their relevancy to each other.

In some aspects of the present disclosure, the processing circuitry modifies the medical query based on user-defined metadata received from the user device.

In some aspects of the present disclosure, prior to transmit the generated response with the caution score, the processing circuitry revises a portion of the generated response that is non-overlapped with the vectorized data when the plagiarism level is beyond the predetermined range. The processing circuitry further compares the revised generated response with the vectorized data to determine the plagiarism level.

In some aspects of the present disclosure, the processing circuitry analyzes the generated response to identify recommendations for operational procedures or invasive/minimally invasive diagnostic procedures. Further, the processing circuitry generates a link for accessing a consent management platform based on the identified recommendations. Further, the processing circuitry generates a risk tolerance score based on responses provided by the patient over the consent management platform to questions related to risks associated with the identified recommendations. Further, the processing circuitry generates a risk matrix that represents probability and severity of risk events associated with the recommended operational procedures or invasive/minimally invasive diagnostic procedures. Further, the processing circuitry stratifies risk to the patient based on the risk tolerance score and the risk matrix. Further, the processing circuitry generates a set of tasks and a questionnaire for the patient based on the stratified risk. Further, the processing circuitry generates a consent form to receive consent of the patient for the operational procedures or the invasive/minimally invasive diagnostic procedure upon completion of the set of tasks and the questionnaire.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a block diagram of a system for generating a response to a query, according to aspects of the present disclosure.

FIG. 2 illustrates a block diagram of an information processing apparatus of FIG. 1 for generating responses to queries, according to an embodiment.

FIG. 3 illustrates an application pipeline for processing and responding to medical queries, in accordance with example embodiments.

FIGS. 4A and 4B illustrate a flowchart of a method for generating a response to a query, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure provides a system, apparatus, and method for generating responses to medical queries. The disclosed system, apparatus, and method utilize advanced artificial intelligence and natural language processing techniques to process and respond to complex medical queries. The system includes a user device and an information processing apparatus. The information processing apparatus comprises processing circuitry and a database. The database is configured to store first data, which includes unstructured patient-based information, and second data, which includes structured environment-based information.

The processing circuitry is configured to receive a medical query from the user device, retrieve the first and second data from the database, and apply a governance layer to the first and second data to remove public health information. The processing circuitry is further configured to vectorize the first and second data to prepare vectorized data, and generate a response to the medical query based on the vectorized data using a large language model (LLM). The processing circuitry also compares the generated response with the vectorized data to determine a plagiarism level and transmits the generated response to the user device when the plagiarism level is within a predetermined range.

In some aspects, the processing circuitry may determine one or more relations between the first data, the second data, and the medical query prior to vectorizing the first and second data. The processing circuitry may also modify the medical query based on user-defined metadata received from the user device. In certain cases, the processing circuitry may generate a caution score when the plagiarism level is beyond the predetermined range and transmit the caution score to the user device.

The disclosed system, apparatus, and method provide several technical advantages, including enhanced data privacy and security, improved accuracy and relevance of responses, increased reliability of generated responses, optimized query processing, flexible data organization and retrieval, and enhanced transparency and user trust.

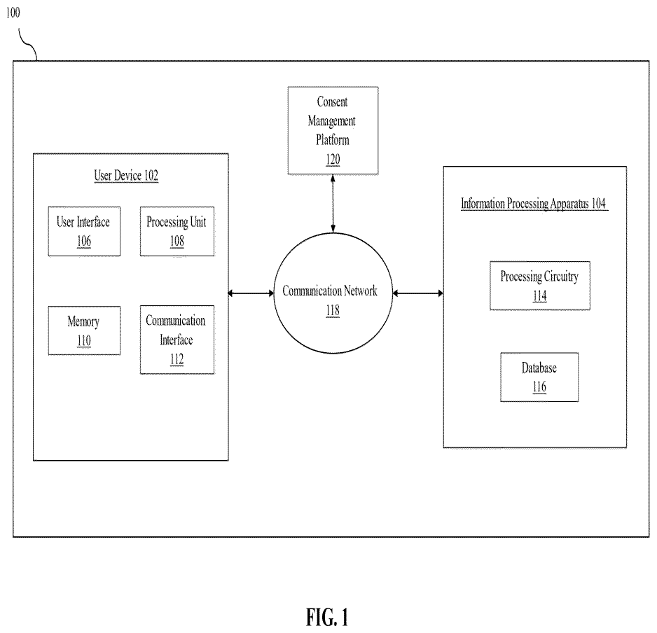

Referring to FIG. 1, the system 100 for generating a response to a medical query includes a user device 102 and an information processing apparatus 104. The user device 102 and the information processing apparatus 104 are communicatively coupled to each other via a communication network 118.

The user device 102 may facilitate a user to interact with the system 100. In some examples of the present disclosure, the user persona may be set as a doctor, a nurse practitioner, a patient, a physician assistant, or a health care worker. The user device 102 may be configured to facilitate the user to login to the system 100. The user interface 106 may be configured to facilitate the user to input or provide a prompt/query to the user device 102. The query may include one or more attributes of the user. The query may include one or more demographic attributes of the patient. The query may include one or more clinical attributes of the patient. The user interface 106 may be further configured so the user can select one or more modes. In preferred examples of this disclosure, the query may be a medical based query. The user device 102 may be configured to receive user defined meta data. The memory 110 may be configured to store the user defined meta data. The query may be modified by the user defined meta data. For example, the user device 102 may be configured to receive the meta data that may be associated with the physician. In such a scenario, the query that may be associated with the physician may be to retrieve information about a patient in a format relevant to the physician. The processing unit 108 may be configured to process the query and the user defined meta data. The processing unit 108 may be configured to, upon processing, modify the query based on the user defined meta data. The processing unit 108 may therefore, may advantageously facilitate to optimize response to the query. Specifically, the processing unit 108, by modifying the query based on the user defined meta data, may advantageously facilitate to make the response to the query very crisp and precise.

In some embodiments of the present disclosure, the user device 102 may be configured to facilitate the user to provide input(s) to register on the system 100. In some other embodiments of the present disclosure, the user device 102 may facilitate the user to enable a password protection for logging-in (i.e., user authentication) to the system 100. In some other embodiments of the present disclosure, the query may be generated by supplementing user identifying information to the query before the prompt is passed on to the large language model.

The user device 102 includes a user interface 106, a processing unit 108, a memory 110, and a communication interface 112.

The user interface 106 of the user device 102 facilitates a user to interact with the system 100. In some aspects, the user interface 106 may be configured to receive a medical query from the user. The user interface 106 may also receive user-defined metadata, which can be stored in the memory 110 of the user device 102. The user-defined metadata may be used to modify the medical query, providing a more personalized and relevant response to the user's query.

In some embodiments of the present disclosure, the user interface 106 may be one of, a touch interface, a mouse, a keyboard, a motion recognition unit, a gesture recognition unit, a voice recognition unit, or the like. Embodiments of the present disclosure are intended to include and/or otherwise cover any type of the first input interface, without deviating from the scope of the present disclosure.

In some embodiments of the present disclosure, the user interface 106 may be configured to enable the user to select and/or provide inputs for registration and/or authentication of the user to use one or more functions in the system 100. The user interface 106 may be further configured to enable the user to provide inputs to enable password protection for logging-in to the system 100. The user interface 106 may include an output interface for displaying (or presenting) any output to the user. Specifically, the output interface may be configured to display or present the response to the query. For example, the output interface may be one of, a digital display, an analog display, a touch screen display, a graphical user interface, a website, a webpage, a keyboard, a mouse, a light pen, an appearance of a desktop, and/or illuminated characters. Embodiments of the present disclosure are intended to include and/or otherwise cover any type of the first output interface including known and/or later developed technologies, without deviating from the scope of the present disclosure.

In some exemplary embodiments of the present disclosure, the user interface 106 may be configured to facilitate the user to select one of, a self-learning mode and a patient care mode.

The processing unit 108 of the user device 102 processes the medical query and the user-defined metadata. Upon processing, the processing unit 108 may modify the medical query based on the user-defined metadata, optimizing the response to the query. The processing unit 108, by modifying the query based on the user-defined metadata, may advantageously facilitate to generate the response to the query very crisp and precise. Examples of the processing unit 108 may include, but not limited to, an application-specific integrated circuit (ASIC) processor, a reduced instruction set computing (RISC) 10 processor, a complex instruction set computing (CISC) processor, a field-programmable gate array (FPGA), a Programmable Logic Control unit (PLC), and the like. Embodiments of the present disclosure are intended to include or otherwise cover any type of processing unit 108 including known, related art, and/or later developed processing units.

The memory 110 of the user device 102 may be configured to store the logic, instructions, circuitry, interfaces, and/or codes of the processing unit 108, data associated with the user device 102, and/or data associated with the system 100. The memory 110 may be configured to store a variety of inputs received from the user. Examples of the memory 110 may include, but not limited to, a Read-Only Memory (ROM), a Random-Access Memory (RAM), a flash memory, a removable storage drive, a hard disk drive (HDD), a solid-state memory, a magnetic storage drive, a Programmable Read Only Memory (PROM), an Erasable PROM (EPROM), and/or an Electrically EPROM (EEPROM). Embodiments of the present disclosure are intended to include or otherwise cover any type of the memory 110 including known, related art, and/or later developed memories.

The communication interface 112 of the user device 102 facilitates communication of the medical query to the information processing apparatus 104 via the communication network 118. The communication network 118 may include suitable logic, circuitry, and interfaces that provide a number of network ports and a number of communication channels for transmission and reception of data related to operations of various entities of the system 100.

The information processing apparatus 104 may be a network of computers i.e., a hardware framework, a software framework, or a combination thereof, that may provide a generalized approach to create a server implementation. Examples of the information processing apparatus 104 may include, but not limited to, personal computers, laptops, mini-computers, mainframe computers, any non-transient and tangible machine that can execute a machine-readable code, cloud-based servers, distributed server networks, or a network of computer systems. The information processing apparatus 104 may be realized through various web-based technologies such as, but not limited to, a Java web-framework, a .NET framework, a personal home page (PHP) framework, or any web-application framework.

The information processing apparatus 104 includes processing circuitry 114 and a database 116. The processing circuitry 114 receives the medical query from the user device 102 via the communication network 118. The processing circuitry 114 retrieves first data and second data from the database 116. The first data may include unstructured patient-based information, and the second data may include structured environment-based information.

The processing circuitry 114 may include suitable logic, instructions, circuitry, interfaces, and/or codes for executing various operations of the system 100. The processing circuitry 114 may be configured to host and enable the user device 102 to execute the operations associated with the system 100 by communicating one or more commands and/or instructions over the communication network 118. The processing circuitry 114 may be configured to receive the query from the user device 102. The processing circuitry 114 may be configured to determine response to the query. Specifically, the processing circuity 114 may be configured to process the query that may be received from the user device 102. The processing circuitry 114, upon processing the query, may be configured to generate the response to the query.

In some exemplary embodiments of the present disclosure, in the self-learning mode, the processing circuitry 114 may be configured to retrieve one or more knowledge sources from the database 116. The one or more knowledge sources may include, but are not limited to, links to various databases (online databases and offline databases), books, notes, and the like. Embodiments of the present disclosure are intended to include and/or otherwise cover any type of the one or more knowledge sources that may be able to provide knowledge to the user, without deviating from the scope of the present disclosure. The user interface 106 may be configured to display the one or more knowledge sources that allow the user to select any knowledge source of the one or more knowledge sources. In the patient care mode, the processing circuitry 114 may be configured to retrieve a guide plan from the database 116. The guide plan may include information pertaining to a patient, diagnosis assistance, treatment assistance, possible steps of treatment of the patient. The guide plan may further represent information pertaining to treatment of the patient that may have symptoms of a disease.

The processing circuitry 114 applies a governance layer to the first data and the second data to remove public health information, ensuring data privacy and security. The processing circuitry 114 then vectorizes the first data and the second data to prepare vectorized data, which enhances the accuracy and relevance of the response to the medical query.

The processing circuitry 114 generates a response to the medical query based on the vectorized data using a large language model (LLM). The generated response is compared with the vectorized data to determine a plagiarism level. If the plagiarism level is within a predetermined range, the processing circuitry 114 transmits the generated response to the user device 102 via the communication network 118. The user device 102, specifically the user interface 106, receives the response to the query from the information processing apparatus 104 and displays the response to the user.

Referring to FIG. 1, the processing circuitry 114 of the information processing apparatus 104 is configured to compare the generated response, which is based on the vectorized data, with the vectorized data itself. This comparison is performed to determine a plagiarism level. The plagiarism level is a measure of the similarity between the generated response and the vectorized data. In some aspects, the plagiarism level may be calculated using various techniques known in the art, such as cosine similarity, Jaccard similarity, or other similarity measures. The plagiarism level may be expressed as a percentage, with a higher percentage indicating a higher level of similarity between the generated response and the vectorized data.

The processing circuitry 114 is further configured to transmit the generated response to the user device 102 when the plagiarism level is within a predetermined range. The predetermined range may be set based on various factors, such as the desired accuracy of the response, the complexity of the medical query, or other factors. In some cases, the predetermined range may be set to a high value, such as 70% or more, to ensure that the generated response is highly similar to the vectorized data. In other cases, the predetermined range may be set to a lower value, such as 50% or less, to allow for more variation in the generated response.

In some aspects, if the plagiarism level is beyond the predetermined range, the processing circuitry 114 may generate a caution score. The caution score is a measure of the discrepancy between the generated response and the vectorized data. A higher caution score indicates a greater discrepancy, which may suggest that the generated response may not be accurate or reliable. The caution score may be calculated using various techniques known in the art, such as error rate calculation, deviation measurement, or other discrepancy measures.

The processing circuitry 114 is further configured to transmit the caution score to the user device 102. The user device 102, specifically the user interface 106, receives the caution score from the information processing apparatus 104 and displays the caution score to the user. The caution score provides the user with an indication of the reliability of the generated response, allowing the user to make informed decisions based on the response. This feature of the system 100 may enhance the transparency of the system and increase user trust in the generated responses.

In some embodiments of the present disclosure, when the plagiarism level is within the predetermined range, the processing circuitry 114 may further be configured to analyze the generated response. Specifically, the processing circuitry 114 may be configured to analyze the generated response to identify recommendations for any operational procedures or any invasive/minimally invasive diagnostic procedure in the generated response by way of an artificial intelligence (AI)/machine learning (ML) technique. When the processing circuitry 114 identifies the recommendations for any operational procedures or any invasive/minimally invasive diagnostic procedure in the generated response, the processing circuitry 114 may further be configured to generate a link. The processing circuitry 114 may be configured to display the link along with the generated response on the user device 102. The link may be a web page link to access a consent management platform 120 by way of the user device 102. The consent management platform 120 may be coupled to the information processing apparatus 104 by way of the communication network. Further, the processing circuitry may be configured to host the consent management platform 120. The patient may receive the link from the user and access the link by way of a user device of the patient. A user interface of the user device of the patient may be configured to enable the patient to select and/or provide inputs for registration and/or authentication of the user to access the consent management platform 120. The user interface of the user device of the patient may be further configured to enable the user to provide inputs to enable password protection for logging-in to the consent management platform 120.

When the patient accesses the consent management platform 120, the user device of the patient may be configured to facilitate the patient to answer a set of questions related to the one or more risks associated with the operational procedures or the invasive/minimally invasive diagnostic procedure recommended to the patient. Further, the processing circuitry 114 may be configured to generate a risk tolerance score for the patient based on the answers provided by the patient. Further, the processing circuitry 114 may be configured to generate a matrix such that columns in the matrix represent probability of occurrence of a risk event during the operational procedures or the invasive/minimally invasive diagnostic procedure and rows of the matrix represent severity associated with the occurrence of the risk event.

Further, the processing circuitry 114 may be configured to stratify the risk for the patient based on the generated risk tolerance score of the patient and the generated matrix. Specifically, the processing circuitry 114 may be configured to stratify the risk for the patient based on the generated risk tolerance score of the patient, the probability of occurrence of a risk event, and the severity associated with the occurrence of the risk event. In the risk stratification, the processing circuitry 114 may be configured to categorize the patient in a category from a plurality of categories based on a risk percentage assigned to a patient. The risk percentage may be assigned to the patient based on the generated risk tolerance score of the patient, the probability of occurrence of the risk event, and the severity associated with the occurrence of the risk event. In an example, the processing circuitry 114 may be configured to categorize the risk percentage assigned to the patient into four categories such that the four categories are (i) >5 percent risk category, (ii) between 1-5 percent risk category, (iii) between 0.1 to 1 percent risk category, and (iv) <0.1 percent risk category.

In some embodiments of the present disclosure, the processing circuitry 114 may be configured to enable the patient to re-answer the set of questions previously answered related to the one or more risks associated with the operational procedures or the invasive/minimally invasive diagnostic procedure by way of the user device of the patient. Based on the answers provided during re-answering the set of questions, the processing circuitry may be configured to the modify the risk tolerance score previously assigned to patient and thereby the risk percentage assigned to the patient.

Further, the processing circuitry 114 may be configured to generate a set of tasks to be completed by the patient and display the set of tasks on the user device of the patient. Specifically, the processing circuitry 114 may be configured to generate the set of tasks depending on the category of the risk percentage assigned to the patient. In an exemplary scenario, the set of tasks generated by the processing circuitry 114 may include, but not limited to, enabling the patient assigned to a high risk category (e.g., >5 percent risk category) to watch a video related to the probability of risk events that may occur during the operational procedures or the invasive/minimally invasive diagnostic procedure. The video may be present in the database 116 and the processing circuitry 114 may be configured to receive the video from the database 116.

Further, the processing circuitry 114 may check if the patient has watched the video completely. When the processing circuitry 114 identifies that the patient has not completely watched the video, the processing circuitry 114 may provide an instruction to the client to resume the video from start and completely watch the video. Further, upon detecting that the patient has completely watched the video, the processing circuitry 114 may generate a questionnaire and display the questionnaire by way of the user device 102 such that the questionnaire is based on the information provided in the video. Further, the processing circuitry 114 may allow the patient to answer the questionnaire using the user device of the patient. If the patient provides wrong answer to one or more questions present in the questionnaire then the processing circuitry 114 may be configured to play the specific part of the video on the user device of the patient corresponding to the question wrongly answered and allow the patient to answer the questionnaire again.

In another exemplary scenario, where the patient may be assigned to a moderate risk category (e.g., between 1-5 percent risk category), the set of tasks generated by the processing circuitry 114 may include, but not limited to, enabling the patient to watch the video related to the probability of risk events that may occur during the operational procedures or the invasive/minimally invasive diagnostic procedure. The processing circuitry 114, upon watching the video, may further generate the questionnaire and allow the user to opt for answering the questionnaire or to skip answering the questionnaire.

In a yet another exemplary scenario, where the patient may be assigned to a mild risk category (e.g., between 0.1 to 1 percent risk category), the set of tasks generated by the processing circuitry 114 may include, but not limited to, enable the patient to opt for watching the video or to skip watching the video.

In yet another exemplary scenario, where the patient may be assigned to a minimum risk category (e.g., <0.1 percent risk category), the set of tasks generated by the processing circuitry 114 may include, but not limited to, enabling the patient to opt for reading a note related to the probability of risk events that may occur during the operational procedures or the invasive/minimally invasive diagnostic procedure or skip reading the note.

Aspects of the present disclosure are intended to include or otherwise cover any set of tasks based on the category of the risk percentage assigned to the patient, without deviating from the scope of the present disclosure.

In some embodiments of the present disclosure, after completion of the set of tasks assigned to the patient, the processing circuitry 114 may be configured to generate a consent form specific to the operational procedures or the invasive/minimally invasive diagnostic procedure using the database 116 and display the consent form on the user device of the patient. Further, the processing circuitry 114 may be configured to facilitate in receiving the consent of the patient for the operational procedures or the invasive/minimally invasive diagnostic procedure by allowing the patient to provide requisite details in the consent form. Further, upon receiving the requisite details in the consent form, the processing circuitry 114 may be configured to enable a user to provide a digital signature of the patient. Upon receiving the digital signature, the processing circuitry 114 may be configured to display a notification on the user device indicating consent verification success.

In some embodiments of the present disclosure, facial recognition may be used to validate the person providing the consent. In some other embodiments of the present disclosure, image capture may be used to validate the person providing the consent. In some other embodiments of the present disclosure, video capture may be used to validate the person providing the consent.

In some embodiments of the present disclosure, the processing circuitry 114 may be configured to allow the user to recertify the consent after a specific period of time. In some embodiments of the present disclosure, the specific period of time may be thirty days. Aspects of the present disclosure are intended to include or otherwise cover, any period of time to recertify the consent, without deviating from the scope of the present disclosure. In some embodiments of the present disclosure, the processing circuitry 114 may advantageously facilitate the patient to recertify the consent form in a manner that involves only verifying the consent form previously submitted by the patient (i.e., submitted before the specific period of time) without a need to provide the requisite details again.

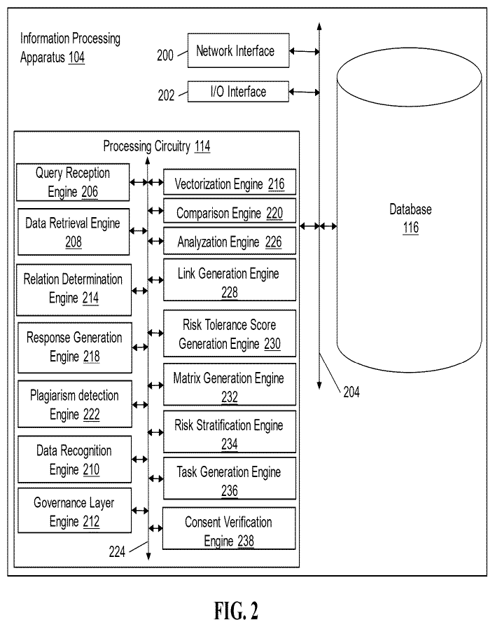

Referring to FIG. 2, the information processing apparatus 104 includes processing circuitry 114 and a database 116. The processing circuitry 114 may include several interconnected components that work together to process queries, generate responses and verify consent. These components include a query reception engine 206, a data retrieval engine 208, a data recognition engine 210, a, a governance layer engine 212, a relation determination engine 214, a vectorization engine 216, a response generation engine 218, a comparison engine 220, a plagiarism detection engine 222, an analyzation engine 226, a link generation engine 228, a risk tolerance score generation engine 230, a matrix generation engine 232, a risk stratification engine 234, a task generation engine 236, and a consent verification engine 238, connected to each other by way of a second communication bus 224.

The query reception engine 206 is configured to receive a medical query from the user device 102. The data retrieval engine 208 is configured to retrieve the first and second data from the database 116. The data recognition engine 210 is configured to recognize the first data and the second data retrieved from the database 116. Specifically, the data recognition engine 210 may be configured to recognize the patient by way of a name entity recognition (NER) technique based on the first and second data. The first data may include unstructured patient-based information, and the second data may include structured environment-based information.

The governance layer engine 212 is configured to apply a governance layer to the first data and the second data to remove public health information from the first data and the second data. This ensures data privacy and security by preventing the public from accessing sensitive patient information.

The relation determination engine 214 is configured to determine one or more relations between the first data, the second data, and the medical query. This allows the system 100 to understand the context of the query and provide a more relevant response.

The vectorization engine 216 is configured to vectorize the first data and the second data to prepare vectorized data. Vectorization is a process that converts data into a format that can be easily processed by machine learning algorithms. This enhances the accuracy and relevance of the response to the medical query. To vectorize the first and second data, the vectorization engine 216 may be configured to prepare a plurality of chunks (hereinafter referred to as “chunks”) from the first and second data. Specifically, the vectorization engine 216 may be configured to separately prepare the plurality of chunks from the first data and the second data. For example, the vectorization engine 216 may be configured to prepare chunks of every 1000 words from words of a textbook. Additionally, the vectorization engine 216 may be configured to separately prepare the plurality of chunks with overlap from the first data and the second data or the vectorized data. In another example, the vectorization engine 216 may be configured to prepare chunks of every 1000 words from words of a textbook with 100 words overlap.

To vectorize the first and second data, the vectorization engine 216 may be configured to organize the chunks of every 1000 words based on their relation to each other. Specifically, the vectorization engine 216, by way of the RAG model architecture, may be configured to set or arrange a sequence of the chunks of every 1000 words based on their relation to each other. For example, the first four sets of every 1000 words may be related to 3 sets of every 1000 words of a different chapter of the textbook. In such a scenario, the vectorization engine 216 may be configured to organize or arrange the first four sets of every 1000 words with the 3 sets of 1000 words of different chapters of the textbook. Thus, the vectorization engine 216 may be configured to prepare a virtual grid by arranging the chunks based on their relevancy/relation to each other. Specifically, the vectorization engine 216 may be configured to prepare the virtual grid by arranging the chunks of the first data and the second data based on their relevancy/relation to each other.

The vectorization engine 216 may further be configured to transfer the query from the Lang chain agent to the RAG model architecture. The vectorization engine 216 may be configured to facilitate the RAG model architecture to retrieve relevant information from the vectorized first and second data. In other words, the vectorization engine 216 may be configured to facilitate the RAG model architecture to retrieve relevant information from the virtual grid. The term “relevant information” as used herein refers to information pertaining to response to the query.

In some exemplary embodiments of the present disclosure, the textbook may be a medical textbook such that the query provided by the user is related to pneumonia. The textbook may describe pneumonia in a first chapter, pathophysiology in a second chapter, and treatment of pneumonia in a third chapter of the textbook. In such a scenario, the vectorization engine 216 may be configured to prepare the chunks based on the relevant excerpts from the first chapter, the second chapter, and the third chapter. The vectorization engine 216 may be configured to prepare the virtual grid based on the first through third chapters. The virtual grid may therefore represent information pertaining to pneumonia, pathophysiology, and treatment of pneumonia. In such a scenario, the vectorization engine 216 may be configured to facilitate the RAG model architecture to retrieve relevant information from the vectorized first and second data. Specifically, the vectorization engine 216 may be configured to facilitate the RAG model architecture to retrieve the relevant information that may represent information about pneumonia, pathophysiology, and treatment of pneumonia.

The response generation engine 218 is configured to generate a response to the medical query based on the vectorized data using a large language model (LLM). The LLM is a machine learning model that is trained on a large amount of text data and can generate human-like text based on the input it receives. The response generation engine 218 is configured to facilitate the LLM model to generate an LLM output. Specifically, response generation engine 218 is configured to facilitate the LLM model to generate the LLM output based on the vectorized first and second data and the query. The response generation engine 218 is further configured to apply the governance layer on the LLM output. Since the LLM output represents very specific information about the patient, hospital, and the like, therefore, the LLM output may not be shown directly to the user. In other words, the LLM output may represent specific information about the patient, hospital, and the like, therefore, the LLM output must not be accessible to the public. The response generation engine 218 is therefore configured to govern the LLM output. Specifically, the response generation engine 218 is configured to remove biasness from the LLM output. The biasness may be a disproportionate weight that may represent favor of or against an idea or thing. The biasness may be innate or learned. The system 100 and the user may develop the biasness in favor or against an individual, a group, or a belief. The biasness, if not addressed, may lead to a suboptimal clinical output. Specifically, the response generation engine 218 may be configured to remove one of, Artificial Intelligence (AI) specific biasness, provider biasness, and medicine biasness, from the LLM output. The response generation engine 218 may therefore facilitate neutralization of the LLM output. In other words, the response generation engine 218 may be configured to make the LLM output independent from any type of the biasness. The biasness in the LLM output may arise due to variation in region, due to variation in diseases, and other factors.

In some embodiments of the present disclosure, the deidentified PHI will be reconstituted with the response output from the LLM within the data privacy wall before providing the response to the user. In some embodiments of the present disclosure, the user interface 106 may be configured to display the LLM output along with the PHIs. In some embodiments of the present disclosure, the patient note will be converted to text, NER performed, PHI governance layer will be applied to generate deidentified data devoid of any PHI, which can be then transmitted and analyzed freely using online tools, the previously removed PHI information will be reconstituted with the response output from the LLM within the data privacy wall before providing the PHI specific response to the user. In an embodiment, the knowledge base or the model may be used for financial or billing purposes.

The comparison engine 220 is configured to compare the generated response with the vectorized data to determine a plagiarism level. This ensures the originality of the generated response and prevents the system from simply copying the input data.

The plagiarism detection engine 222 is configured to detect any instances of plagiarism in the generated response. If the plagiarism level is beyond a predetermined range, the system generates a caution score and transmits it to the user device 102. This provides the user with an indication of the reliability of the generated response, allowing the user to make informed decisions based on the response.

In some aspects, the processing circuitry 114 may further be configured to modify the medical query based on user-defined metadata received from the user device 102. This allows the system to provide a more personalized and relevant response to the user's query.

In some cases, the processing circuitry 114 may be configured to revise a portion of the generated response that is not overlapped with the vectorized data when the plagiarism level is beyond the predetermined range. This ensures that the generated response is not simply a copy of the input data and provides a more accurate and relevant response to the medical query.

In some aspects, the processing circuitry 114 may be configured to generate a caution score when the plagiarism level is beyond the predetermined range and transmit the caution score to the user device 102. This provides the user with an indication of the reliability of the generated response, allowing the user to make informed decisions based on the response.

The analyzation engine 226 may be configured to receive the plagiarism level from the plagiarism detection engine 222 and analyze the generated response when the plagiarism level is within a predetermined range. Specifically, the analyzation engine 226 may be configured to analyze the generated response to identify recommendations for any operational procedures or any invasive/minimally invasive diagnostic procedure in the generated response by way of an artificial intelligence (AI)/machine learning (ML) technique.

The link generation engine 228 may be configured to receive the identified recommendations for any operational procedures or any invasive/minimally invasive diagnostic procedure in the generated response and display a link to the patient by way of the user device of the patient to access the consent management platform 120. In some embodiments of the present disclosure, the link generation engine 228 may be configured to enable the patient to select and/or provide inputs for registration and/or authentication to access the consent management platform 120.

The risk tolerance score generation engine 230 may be coupled to the link generation engine 228. When the patient accesses the consent management platform 120 after registration/authentication, the risk tolerance score generation engine 230 may be configured to facilitate the patient to answer a set of questions related to the one or more risks associated with the operational procedures or the invasive/minimally invasive diagnostic procedure recommended to the patient. Further, the risk tolerance score generation engine 230 may be configured to generate a risk tolerance score for the patient based on the answers provided by the patient.

The matrix generation engine 232 may be configured to generate a matrix such that columns in the matrix represent probability of occurrence of a risk event during the operational procedures or the invasive/minimally invasive diagnostic procedure and rows of the matrix represent severity associated with the occurrence of the risk event.

The risk stratification engine 234 may be configured to receive the generated risk tolerance score and the generated matrix from the risk tolerance score generation engine 230 and the matrix generation engine 232, respectively. Further, the risk stratification engine 234 may be configured to stratify the risk for the patient based on the generated risk tolerance score of the patient, the probability of occurrence of a risk event, and the severity associated with the occurrence of the risk event. Further, the risk stratification engine 234 may be configured to categorize the patient in a category from the plurality of categories based on the risk percentage assigned to a patient. The risk percentage may be assigned to the patient based on the generated risk tolerance score of the patient, the probability of occurrence of the risk event, and the severity associated with the occurrence of the risk event.

The task generation engine 236 may be configured to receive the category of the risk percentage assigned to the patient. Further, task generation engine 236 may be configured to generate a set of tasks to be completed by the patient and display the set of tasks on the user device of the patient based on the category of the risk percentage assigned to the patient. In some embodiments of the present disclosure, the set of tasks may include watching a video, listening to an audio, reading a note or any combination thereof, depending on the category of the risk percentage assigned to the patient. In some embodiments of the present disclosure, the task generation engine 236 may be configured to generate a questionnaire to be answered by the patient, upon completion of the set of tasks. In some cases, the task generation engine 236 may be configured to re-assign the set of tasks to the patients, if one or more questions in the questionnaire is wrongly answered by the patient.

The consent verification engine 238 may be configured to receive the information regarding the set of tasks and the questionnaire. Further, upon the completion of the set of tasks and the questionnaire, the consent verification engine 238 may be configured to generate a consent form using the database 116 such that the consent form is specific to the operational procedures, or the invasive/minimally invasive diagnostic procedure recommended to the patient. Further, the consent verification engine 238 may be configured to display the generated consent form on the user device of the patient and allow the patient to provide requisite details and/or signature of the patient in the consent form. Furthermore, the consent verification engine 238 may be configured to display the notification on the user device indicating consent verification success, upon receiving the requisite details and/or signature in the consent form. In some embodiments of the present disclosure, the consent verification engine 238 may be configured to allow the patient to recertify the consent after a specific period of time has passed.

Thus, the information processing apparatus 104, by way of the processing circuitry 114, provides a robust and efficient mechanism for processing medical queries, generating accurate and relevant responses, and receiving the consent from the patient. The use of a governance layer ensures data privacy and security, while the use of vectorization and a large language model enhances the accuracy and relevance of the generated responses. The system also provides a mechanism for detecting plagiarism and revising the generated response, ensuring the originality and reliability of the responses.

The database 116 of the information processing apparatus 104 is configured to store first data comprising unstructured patient-based information and second data comprising structured environment-based information. The database 116 may include suitable logic, circuitry, interfaces, and/or code, executable by the circuitry, to perform one or more operations. For example, the database 116 may be configured to store the first data and the second data in a structured format that allows for efficient retrieval and processing of the data. The database 116 may also be configured to manage the storage and retrieval of the data in a secure and efficient manner, ensuring data privacy and security. Further, the database 116 may store the set of tasks and the questionnaire to be completed by the patient.

The information processing apparatus 104 also includes a network interface 200 and an I/O interface 202. The network interface 200 is configured to facilitate communication between the information processing apparatus 104 and other devices, such as the user device 102, over a communication network. The I/O interface 202 is configured to facilitate input and output operations within the information processing apparatus 104, such as receiving queries from the user device 102 and transmitting responses to the user device 102. The processing circuitry 114, the database 116, the network interface 200, and the I/O interface 202 may communicate to each other by way of a first communication bus 204.

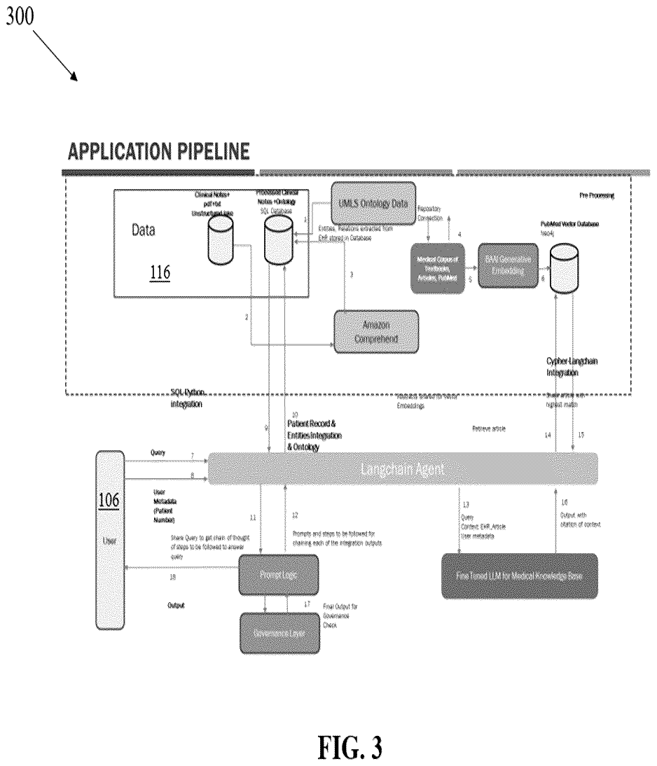

Referring to FIG. 3, the application pipeline 300 for processing and responding to medical queries is illustrated. The application pipeline 300 includes a database 116, a user interface 106, and various processing modules. The database 116 stores multiple data sources, including clinical notes, medical ontology data (UMLS Ontology Data), and a knowledge database. These data sources provide the foundation for the system's medical knowledge and information retrieval capabilities.

The user interface 106 of the user device 102 facilitates a user to interact with the system 100. In some aspects, the user interface 106 may be configured to receive a medical query from the user. The user interface 106 may also receive user-defined metadata, which can be stored in the memory 110 of the user device 102. The user-defined metadata may be used to modify the medical query, providing a more personalized and relevant response to the user's query.

The application pipeline 300 includes several processing modules that work together to analyze queries and generate responses. These modules include Amazon Comprehend, SAM Generative Embedding, Langchain Agent, Fine Tuned LLM for Medical Knowledge Base, and a Governance Layer.

Amazon Comprehend is a natural language processing (NLP) service that uses machine learning to find insights and relationships in text. In some aspects, Amazon Comprehend may be used for natural language processing tasks in the system 100. Specifically, Amazon Comprehend may be used to analyze the medical query received from the user and extract relevant information.

Segment Anything Model (SAM) Generative Embedding is a technique for generating embeddings of medical text. Embeddings are a type of word representation that allows words with similar meaning to have a similar representation. In some aspects, SAM Generative Embedding may be used to generate embeddings of the first data and the second data. This enhances the accuracy and relevance of the response to the medical query.

The Langchain Agent serves as the central processing unit for handling user queries and coordinating responses. In some aspects, the Langchain Agent may be configured to receive the medical query from the user device 102, process the query, and coordinate the generation of the response.

The Fine Tuned LLM for Medical Knowledge Base is a language model specifically trained on medical data to provide accurate responses. In some aspects, the Fine Tuned LLM for Medical Knowledge Base may be used to generate a response to the medical query based on the vectorized data.

The Governance Layer ensures compliance and proper handling of sensitive medical information. In some aspects, the Governance Layer may be applied to the first data and the second data to remove public health information, ensuring data privacy and security.

Thus, the application pipeline 300 integrates various data sources and processing techniques to provide accurate and context-aware responses to medical queries. The use of advanced natural language processing techniques, such as Amazon Comprehend and SAM Generative Embedding, enhances the accuracy and relevance of the responses. The use of a governance layer ensures data privacy and security. The use of a large language model, such as the Fine Tuned LLM for Medical Knowledge Base, provides human-like responses to medical queries.

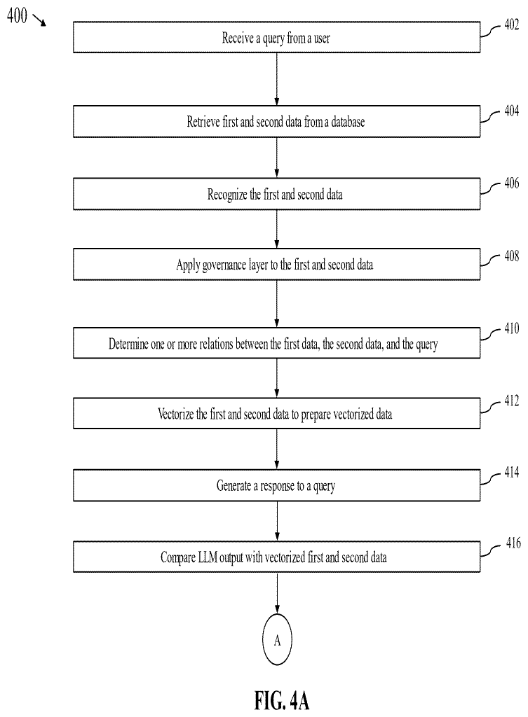

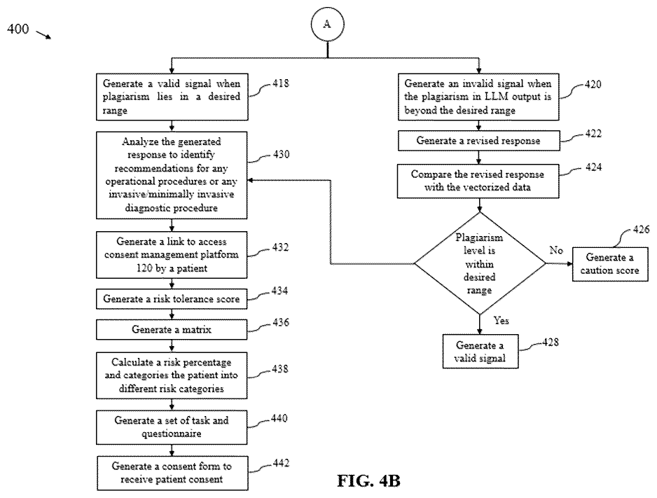

Referring to FIGS. 4A and 4B, the method 400 for generating a response to a query is illustrated. The method 400 begins with step 402, where the system 100 receives a query from a user. The query may be a medical query related to a specific patient or a general medical question. The query may be received via the user interface 106 of the user device 102.

At step 404, the processing circuitry 114 retrieves first data and second data from the database 116. The first data may include unstructured patient-based information, such as medical history, symptoms, and test results. The second data may include structured environment-based information, such as hospital records, treatment protocols, and medical guidelines. The first data and the second data may be retrieved based on the query received from the user.

At step 406, the processing circuitry 114 recognizes the first data and the second data. The recognition process may involve identifying relevant information in the first data and the second data based on the query. The recognition process may be performed using various techniques known in the art, such as natural language processing, machine learning, or other data analysis techniques.

At step 408, the processing circuitry 114 applies a governance layer to the first data and the second data. The governance layer may be configured to remove public health information (PHI) from the first data and the second data. This ensures data privacy and security by preventing unauthorized access to sensitive patient information.

At step 410, the processing circuitry 114 determines one or more relations between the first data, the second data, and the query. The relations may be determined based on various factors, such as the content of the query, the content of the first data and the second data, and the context of the query. The relations may be used to enhance the relevance and accuracy of the response to the query.

At step 412, the processing circuitry 114 vectorizes the first data and the second data to prepare vectorized data. The vectorization process may involve converting the first data and the second data into a format that can be easily processed by machine learning algorithms. The vectorization process may enhance the accuracy and relevance of the response to the query.

At step 414, the processing circuitry 114 generates a response to the query based on the vectorized data. The response may be generated using a large language model (LLM). The LLM may be a machine learning model that is trained on a large amount of text data and can generate human-like text based on the input it receives.

At step 416, the processing circuitry 114 compares the generated response with the vectorized data to determine a plagiarism level. The plagiarism level may be a measure of the similarity between the generated response and the vectorized data. If the plagiarism level is within a predetermined range, the processing circuitry 114 generates a valid signal at step 418, indicating that the generated response is valid and can be transmitted to the user device 102.

If the plagiarism level is beyond the predetermined range, the processing circuitry 114 generates an invalid signal at step 420. In such a case, at step 422, the processing circuitry 114 may revise a portion of the generated response that is not overlapped with the vectorized data. The revision process may involve various techniques known in the art, such as text rewriting, paraphrasing, or other text generation techniques. At step 424, the revised response may then be compared with the vectorized data again to determine a new plagiarism level. At step 4226, If the new plagiarism level is within the predetermined range, the processing circuitry 114 generates a valid signal, and the revised response is transmitted to the user device 102. At step 428, If the new plagiarism level is still beyond the predetermined range, the processing circuitry 114 may generate a caution score and transmit it to the user device 102. The caution score provides the user with an indication of the reliability of the generated response, allowing the user to make informed decisions based on the response.

At step 430, when the valid signal is generated, the processing circuitry 114 may analyze the generated response to identify recommendations for any operational procedures or any invasive/minimally invasive diagnostic procedure in the generated response by way of the artificial intelligence (AI)/machine learning (ML) technique.

At step 432, when the processing circuitry 114 identifies the recommendations for any operational procedures or any invasive/minimally invasive diagnostic procedure in the generated response, the processing circuitry 114 may further be configured to generate the link. The link may be the web page link to access the consent management platform 120 that may be coupled to the information processing apparatus 104 by way of the communication network.

At step 434, upon accessing the consent management platform 120, the processing circuitry 114 may facilitate the patient to answer a set of questions related to the one or more risks associated with the operational procedures or the invasive/minimally invasive diagnostic procedure recommended to the patient and thereby generate the risk tolerance score for the patient based on the answers provided by the patient.

At step 436, the processing circuitry 114 may be configured to generate the matrix such that columns in the matrix represent probability of occurrence of a risk event during the operational procedures or the invasive/minimally invasive diagnostic procedure and rows of the matrix represent severity associated with the occurrence of the risk event.

At step 438, the processing circuitry 114 may be configured to stratify the risk for the patient based on the generated risk tolerance score of the patient and the generated matrix. In the risk stratification, the processing circuitry 114 may be configured to categorize the patient in a category from a plurality of categories based on a risk percentage assigned to a patient.

At step 440, the processing circuitry 114 may generate a set of tasks to be completed by the patient and may generate a questionnaire to be answered by the patient upon completion of the generated set of tasks.

At step 442, the processing circuitry 114 may generate a consent form specific to the operational procedures or the invasive/minimally invasive diagnostic procedure using the database 116. Further, at step 442, the processing circuitry 114 may allow the patient to provide requisite details and/or signature in the generated consent form thereby facilitating in receiving the consent of patient.

In some aspects of the present disclosure, the method 400 may include steps in which the processing circuitry 114 may be configured to allow the user to recertify the consent after a specific period of time and thereby facilitating in successful verification of consent of the patient.

Thus, the method 400 provides a robust and efficient mechanism for generating responses to queries and consent verification. The method 400 utilizes advanced techniques such as data recognition, data vectorization, and plagiarism detection to ensure the accuracy, relevance, and originality of the generated responses. The method 400 also ensures data privacy and security by applying a governance layer to the data. The method 400 further provides a mechanism for revising the generated response and generating a caution score when the plagiarism level is beyond a predetermined range, enhancing the transparency and reliability of the generated responses. The method 400 further provides a reliable approach to consent verification.

In some aspects, the system 100 may be applied to areas beyond medical query response generation and consent verification. For instance, the system 100 may be utilized for financial or billing purposes in healthcare settings. The system 100, by way of the processing circuitry 114, may be configured to process queries related to financial or billing information. The first data and the second data retrieved from the database 116 may include financial records, billing records, insurance information, and other related data. The processing circuitry 114 may apply the same techniques of data recognition, relation determination, vectorization, and response generation to generate responses to financial or billing queries. This may include generating billing statements, calculating costs, determining insurance coverage, and other financial operations. The generated responses may then be transmitted to the user device 102, providing users with accurate and relevant financial or billing information. This application of the system 100 may enhance the efficiency and accuracy of financial operations in healthcare settings, thereby improving financial management and reducing administrative burdens.

In some aspects of the present disclosure, the system 100 and the method 400 may include inputting the query that may be based on one or more scientific papers. The query may be generated and/or validated by the processing circuitry 114 by way of LLM. Specifically, the processing circuitry 114 may be configured to generate the query from the one or more scientific papers in the form of a set of questions by way of LLM such that the set of questions may include a plurality of multiple choice questions or the like. Further, the processing circuitry 114 may be configured to compare the generated set of questions with the one or more scientific papers to determine the plagiarism level. Further, the processing circuitry 114 may be configured to determine the plagiarism level by checking for textual plagiarism and for semantic plagiarism. For checking the textual plagiarism, the processing circuitry 114 may be configured to check for words/sentences that are exactly and/or explicitly present in the one or more scientific papers. For checking the semantic plagiarism, the processing circuitry 114 may be configured to check for contents from the one or more scientific papers that match with the meaning of the query. When the processing circuitry 114 determines that the plagiarism level is within the predefined range, the processing circuitry may be configured to provide a critique and explanation for the correct answer and the incorrect options. When the processing circuitry 114 determines that the plagiarism level is not within the predefined range, the processing circuitry 114 may be configured to generate an invalid signal to regenerate the set of questions for checking plagiarism. Further, the processing circuitry 114 may be configured to summarize a plurality of learning points and thereby provide a comprehensive summary of the one or more scientific papers.

In an exemplary embodiment of the present disclosure, the processing circuitry 114 may include a plurality of Agentic Artificial Intelligence (Agentic AI) agents such that the plurality of Agentic AI agents may be configured to perform tasks similar to the tasks performed by the engines 206-238 (as explained in FIG. 2).

In another exemplary embodiment of the present disclosure, the system 100 may be coupled to an overarching Agentic Artificial Intelligence (Agentic AI) system that may include the plurality of Agentic AI agents. The overarching Agentic Artificial Intelligence (Agentic AI) system may provide an added assurance mechanism for informational integrity and data governance. The overarching Agentic Artificial Intelligence (Agentic AI) system may facilitate pre-empting any external criticism by introducing the plurality of Agentic AI agents that proactively identify, address and overcome any possibility of a sub-optimal outcome. Specifically, the plurality of Agentic AI agents may be configured to perform: (a) response preparation (b) criticizing and challenging the response (c) adjudication of disputes on scientific evidence and logic, and (d) as a public welfare maximizer in line with classical economics theory. Thus, the framework of the overarching Agentic Artificial Intelligence (Agentic AI) system may enhance the maturity of product and solution by way of adversarial back and forth between the agentic AI agents, game theoretical strategizing, decision making, and supremacy of overarching public good maximization. In some embodiments, the output from the overarching Agentic Artificial Intelligence (Agentic AI) system may be checked for textual and semantic plagiarism check to validate output in a given range. In some embodiments, textual plagiarism can be more than 50% and semantic plagiarism more than 75% to qualify for a successful response.

Thus, the system, the apparatus, and the method of the present disclosure provide enhanced data privacy and security through the application of a governance layer that removes public health information from patient data before processing. Further, the system, the apparatus, and the method of the present disclosure provides improved accuracy and relevance of responses through vectorization of both unstructured patient data and structured environmental data, allowing for more comprehensive analysis. Furthermore, the system, the apparatus, and the method of the present disclosure provides increased reliability of generated responses by implementing a plagiarism detection mechanism that compares the output against the vectorized data. Furthermore, the system, the apparatus, and the method of the present disclosure provide optimized query processing through the use of user-defined metadata to modify and refine medical queries. Furthermore, the system, the apparatus, and the method of the present disclosure provide flexible data organization and retrieval via the creation of a virtual grid of data chunks arranged based on relevance and relationships. Furthermore, the system, the apparatus, and the method of the present disclosure provide enhanced transparency and user trust through the generation of caution scores when plagiarism levels exceed predetermined thresholds.

The present disclosure for medical query processing combines advanced natural language processing techniques with robust data management and security measures, resulting in a system that can provide more accurate, relevant, and trustworthy responses to complex medical queries while maintaining patient privacy and data integrity.

A number of implementations have been described. Nevertheless, it will be understood that various modifications may be made without departing from the spirit and scope of the disclosure. Accordingly, other implementations are within the scope of the following claims.

1. A method for generating a response to a medical query, the method comprising:
receiving, by processing circuitry, a medical query from a user device coupled to the processing circuitry;
retrieving, by the processing circuitry, first data and second data from a database coupled to the processing circuitry, wherein the first data comprises unstructured patient-based information and the second data comprises structured environment-based information;
applying, by the processing circuitry, a governance layer to the first data and the second data to remove public health information;
vectorizing, by the processing circuitry, the first data and the second data to prepare vectorized data;
generating, by the processing circuitry, a response to the medical query based on the vectorized data using a large language model (LLM);
comparing, by the processing circuitry, the generated response with the vectorized data to determine a plagiarism level;
transmitting, by the processing circuitry, the generated response to the user device when the plagiarism level is within a predetermined range; and
transmitting, by the processing circuitry, the generated response with a caution score to the user device such that the caution score is generated when the plagiarism level is beyond the predetermined range.

2. The method of claim 1, further comprising:
determining, by the processing circuitry, one or more relations between the first data, the second data, and the medical query prior to vectorizing the first data and the second data.

3. The method of claim 1, wherein for vectorizing the first data and the second data, the method further comprising:
preparing, by the processing circuitry, a plurality of chunks from the first data and the second data; and
organizing, by the processing circuitry, the plurality of chunks based on relation of the first data and the second data to each other.

4. The method of claim 1, further comprising:
modifying, by the processing circuitry, the medical query based on user-defined metadata received from the user device.

5. The method of claim 1, prior to transmitting the generated response with the caution score, the method further comprising:
revising, by the processing circuitry, a portion of the generated response that is non-overlapped with the vectorized data when the plagiarism level is beyond the predetermined range; and
comparing, by the processing circuitry, the revised generated response with the vectorized data to determine the plagiarism level.

6. The method of claim 1, further comprising:
analyzing, by the processing circuitry, the generated response to identify recommendations for operational procedures or invasive/minimally invasive diagnostic procedures;
generating, by the processing circuitry, a link for accessing a consent management platform based on the identified recommendations;
generating, by the processing circuitry, a risk tolerance score based on responses provided by the patient over the consent management platform to questions related to risks associated with the identified recommendations;
generating, by the processing circuitry, a risk matrix representing probability and severity of risk events associated with the recommended operational procedures or invasive/minimally invasive diagnostic procedures;
stratifying, by the processing circuitry, risk to the patient based on the risk tolerance score and the risk matrix;
generating, by the processing circuitry, a set of tasks and a questionnaire for the patient based on the stratified risk; and
generating, by the processing circuitry, a consent form to receive consent of the patient for the operational procedures or the invasive/minimally invasive diagnostic procedure upon completion of the set of tasks and the questionnaire.

7. An information processing apparatus for generating a response to a medical query, the information apparatus comprising:
a database configured to store first data comprising unstructured patient-based information and second data comprising structured environment-based information; and
processing circuitry that is coupled to the database, and configured to:
receive a medical query from a user device coupled to the information processing apparatus;
retrieve the first data and the second data from the database;
apply a governance layer to the first data and the second data to remove public health information;
vectorize the first data and the second data to prepare vectorized data;
generate a response to the medical query based on the vectorized data using a large language model (LLM);
compare the generated response with the vectorized data to determine a plagiarism level; and
transmit the generated response to the user device when the plagiarism level is within a predetermined range; and
transmit the generated response with a caution score to the user device such that the caution score is generated when the plagiarism level is beyond the predetermined range.

8. The information processing apparatus of claim 6, wherein the processing circuitry is further configured to determine one or more relations between the first data, the second data, and the medical query prior to vectorizing the first data and the second data.

9. The information processing apparatus of claim 6, wherein to vectorize the first data and the second data, the processing circuitry is configured to:
prepare a plurality of chunks from the first data and the second data; and
organize the plurality of chunks based on relation of the first data and the second data to each other.

10. The information processing apparatus of claim 8, wherein to organize the plurality of chunks, the processing circuitry is configured to arrange the plurality of chunks into a virtual grid based on relevancy of the plurality of chunks to each other.

11. The information processing apparatus of claim 6, wherein the processing circuitry is further configured to modify the medical query based on user-defined metadata received from the user device.

12. The information processing apparatus of claim 6, wherein prior to transmit the generated response with the caution score, the processing circuitry is further configured to:
revise a portion of the generated response that is non-overlapped with the vectorized data when the plagiarism level is beyond the predetermined range; and
compare the revised generated response with the vectorized data to determine the plagiarism level.

13. The information processing apparatus of claim 6, wherein the processing circuitry is further configured to:
analyze the generated response to identify recommendations for operational procedures or invasive/minimally invasive diagnostic procedures;
generate a link for accessing a consent management platform based on the identified recommendations;
generate a risk tolerance score based on responses provided by the patient over the consent management platform to questions related to risks associated with the identified recommendations;
generate a risk matrix that represents probability and severity of risk events associated with the recommended operational procedures or invasive/minimally invasive diagnostic procedures;
stratify risk to the patient based on the risk tolerance score and the risk matrix;
generate a set of tasks and a questionnaire for the patient based on the stratified risk; and
generate a consent form to receive consent of the patient for the operational procedures or the invasive/minimally invasive diagnostic procedure upon completion of the set of tasks and the questionnaire.

14. A system for generating a response to a medical query, the system comprising:
a user device configured to transmit a medical query and receive a response corresponding to the medical query;
an information processing apparatus comprising:
a database configured to store first data comprising unstructured patient-based information and second data comprising structured environment-based information; and
processing circuitry that is coupled to the database, and configured to:
receive a medical query from a user device coupled to the information processing apparatus;
retrieve the first data and the second data from the database;
apply a governance layer to the first data and the second data to remove public health information;
vectorize the first data and the second data to prepare vectorized data;
generate a response to the medical query based on the vectorized data using a large language model (LLM);
compare the generated response with the vectorized data to determine a plagiarism level; and
transmit the generated response to the user device when the plagiarism level is within a predetermined range; and
transmit the generated response with a caution score to the user device such that the caution score is generated when the plagiarism level is beyond the predetermined range.

15. The system of claim 13, wherein the processing circuitry is further configured to determine one or more relations between the first data, the second data, and the medical query prior to vectorizing the first data and the second data.

16. The system of claim 13, wherein to vectorize the first data and the second data, the processing circuitry is configured to:
prepare a plurality of chunks from the first data and the second data; and
organize the plurality of chunks based on relation of the first data and the second data to each other.

17. The system of claim 15, wherein to organize the plurality of chunks, the processing circuitry is configured to arrange the plurality of chunks into a virtual grid based on relevancy of the plurality of chunks to each other.

18. The system of claim 13, wherein the processing circuitry is further configured to modify the medical query based on user-defined metadata received from the user device.

19. The system of claim 13, wherein prior to transmit the generated response with the caution score, the processing circuitry is further configured to:
revise a portion of the generated response that is non-overlapped with the vectorized data when the plagiarism level is beyond the predetermined range; and
compare the revised generated response with the vectorized data to determine the plagiarism level.

20. The system of claim 13, wherein the processing circuitry is further configured to:
analyze the generated response to identify recommendations for operational procedures or invasive/minimally invasive diagnostic procedures;
generate a link for accessing a consent management platform based on the identified recommendations;
generate a risk tolerance score based on responses provided by the patient over the consent management platform to questions related to risks associated with the identified recommendations;
generate a risk matrix that represents probability and severity of risk events associated with the recommended operational procedures or invasive/minimally invasive diagnostic procedures;
stratify risk to the patient based on the risk tolerance score and the risk matrix;
generate a set of tasks and a questionnaire for the patient based on the stratified risk; and
generate a consent form to receive consent of the patient for the operational procedures or the invasive/minimally invasive diagnostic procedure upon completion of the set of tasks and the questionnaire.
